(19) United States
(12) Patent Application Publication
(10) Pub. No.: US 2009/0325253 A1
(43) Pub. Date: Dec. 31, 2009

(54) METHODS AND SYSTEMS FOR PRODUCTION OF BIOFUELS AND BIOENERGY PRODUCTS FROM SEWAGE SLUDGE, INCLUDING RECALCITRANT SLUDGE

(76) Inventors: Miguel ASCON, Baltimore, MD (US); Dolores ASCON, Baltimore, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/429,858

(22) Filed: Apr. 24, 2009

Related U.S. Application Data
(60) Provisional application No. 61/125,490, filed on Apr. 25, 2008.

(51) Int. Cl. C12P 7/08; C12M 1/00

(52) U.S. Cl. 435/163; 435/289.1; 435/293.1

(57) ABSTRACT

The present invention provides methods and systems (SLUDFUEL system) for producing biofuel and bioenergy products using, as starting raw material, municipal, industrial, and/or farm sewage sludge, including recalcitrant sludge containing high concentrations of heavy metals, and produced after waste treatment. In accordance with the invention, municipal, industrial, and farm sewage sludge, including recalcitrant sludge, can serve as a carbon source to support the metabolism of synthetic microorganisms to produce biofuels and bioenergy products.

### PRIORITY

This application claims priority to U.S. Provisional Application No. 61/125,490, filed Apr. 25, 2008, which is hereby incorporated by reference in its entirety.

### FIELD OF THE INVENTION

The present invention relates to the production of biofuels and bioenergy products, including biodiesel, ethanol, butanol, methane, hydrogen, and methanol among others. The present invention relates to the production of such fuels from municipal, industrial, and farm sewage sludge, and particularly recalcitrant sludge, for example, which may contain high concentrations of heavy metals, and which may be deleterious when disposed in the environment.

### BACKGROUND OF THE INVENTION

There is an ever-increasing demand for renewable biofuels and bioenergy products as an alternative to fossil fuels. Biofuels are currently produced from, for example, various cellulosic materials and sugar-based plants, including sugarcane, beets, corn, rice, potatoes (among others), as well as wood chips. While the process is straight forward, producing biofuels and bioenergy products from these materials is, overall, inefficient and expensive given the cost of the source materials, and tends to drive up the price of food. Further, the current raw material sources for production of biofuel will not be sufficient to meet the escalating demands.

The U.S. population generates around 8.6 million dry metric tons of sludge annually, that is, approximately 13 billions pounds (dry basis) of sludge. Disposal of this enormous amount of sludge without substantial impact on the environment is an ongoing challenge. For example, in the state of Maryland, more than 700,000 wet tons of sewage sludge is generated each year. Around 50 percent of this sewage sludge is applied to agricultural land, 18 percent is composted or pelletized and made into a commercial soil supplement, and 21 percent is used for land reclamation such as restoring surface mines. The remaining 11 percent is disposed in landfills or incinerated.

In addition, in the United States approximately 230 million tons (dry matter) of animal waste (manure) are generated every year. Unsafe and improper disposal of decomposable animal waste causes substantial environmental pollution, including surface and groundwater contamination, odors, dust, and methane and ammonia emission.

Processing and/or disposal of municipal, industrial, and farm sewage waste (e.g., sludge) is costly, and has an enormous impact on the environment as well as on the public health. For example, with respect to biosolids spread or applied to the soil, such fertilizers may have a high content of pathogenic organisms, and indeed, there have been reports of infection and death associated with contamination of the soil. Further, the biosolids used as fertilizers often have a high content of heavy metals, as well as high contents of organic material (20-40%) that are not appropriate to fertilize the soil. Optimal compost materials have around 5-7% of organic matter, and do not contain significant amounts of heavy metals.

Further, most industrial facilities discharge their wastewater to a local municipal treatment facility. Due to Federal regulations regarding wastewater pretreatment, and due to fees charged by local wastewater treatment facilities, many industrial facilities pretreat their wastewater. The EPA sets pretreatment standards in 40 CFR Part 403 “General Pretreatment Regulations for Existing and New Sources of Pollution”. These regulations apply to more than 40 specified industries. Pretreatment standards also apply to the discharge of more than 120 specific pollutants. Most treatment facilities charge industrial users a fee based on the amount and types of pollutants discharged. Considerations that affect the amount of pretreatment a particular industrial facility will undergo, include: the pollutants to be removed, space availability for equipment, and seasonal flow and pollutant concentration.

Treating and disposing of sewage sludge, including management of the solids and concentrated contaminants, is technically complex given the nature of the material. Sewage sludge is made up largely of those substances responsible for the offensive character of untreated wastewater. The composition of sludge can be roughly characterized by six groups of components: a) non-toxic organic carbon compounds, Kjeldahl-N and/or phosphorus containing components; b) toxic pollutants, such as polychlorinated biphenyls (PCBs), polycyclic aromatic hydrocarbons (PAHs), dioxins, pesticides, endocrine disrupters, linear alkyl-sulfonates, nonyl-phenols, etc; c) heavy metals, such as Zn, Pb, Cu, Cr, Ni, Cd, and Hg (each of which may vary from more than 1000 ppm to less than 1 ppm); d) pathogens and other microbiological pollutants; f) inorganic compounds such as silicates, aluminates, calcium and magnesium containing compounds; and g) water, varying from a few percent to more than ninety five percent.

Recalcitrant sludge as may result from primary waste water treatment and anaerobic and/or aerobic digestion of raw sludge, may contain particularly large concentrations of heavy metals and other toxic substances, and is difficult to treat further or to apply for a useful purpose. Nevertheless, as demonstrated herein, the nitrogen, phosphorus, and organic carbon-containing compounds in sludge (including recalcitrant sludge) are valuable resources that, if properly processed from sludge, can be used for the synthesis of biofuel and bioenergy products.

Methods and systems are needed for recovering valuable components of sewage sludge, including recalcitrant sludge, to help satisfy energy needs, while simultaneously reducing the impact of such waste on the environment and the health of the population.

### SUMMARY OF THE INVENTION

The present invention provides methods and systems for producing biofuel and bioenergy products using, as starting raw material, municipal, industrial, and/or farm sewage sludge, including recalcitrant sludge produced after treatment of raw sludge. In accordance with the invention, municipal, industrial, and farm sewage sludge, including recalcitrant sludge, can serve as a carbon source to support the metabolism of fermentative, methogenic, and/or photosynthetic microorganisms to produce biofuels and bioenergy products.

Sludge is generated by the treatment of waste water. Due to the physical-chemical processes involved in the treatment, the sludge tends to concentrate heavy metals and poorly biodegradable organic compounds, as well as potentially pathogenic organisms present in waste waters. Sludge does also contain, however, valuable organic matter the use of which is hindered by the presence of heavy metals, pathogenic organisms, and/or chemical pollutants. In accordance with the invention, input sludge material is subjected to bioleaching with acid-producing, sulfur-oxidizing bacteria, to solubilize and extract heavy metals and reduce or eliminate pathogens, followed by the biosynthesis of biofuel and bioenergy products from the organic material. The biofuel or bioenergy products may include ethanol, methanol, butanol, biodiesel, methane, and hydrogen, among others.

In one aspect, the present invention provides a method for generating one or more biofuels or bioenergy products. The method comprises treating sludge with the metal leaching action of acid-producing and sulfur-oxidizing bacteria to extract heavy metals (e.g., bioleaching), and to thereby reduce or eliminate pathogens by the resulting low pH. Heavy metals, which are solubilized by the low pH, are removed, for example, by precipitation from the aqueous (liquid) phase. The solid biomass (or “treated sludge”) becomes more uniform and standardized as a result of the bioleaching treatment, and also becomes a suitable carbon source for the synthesis of one or more biofuels from the organic material by fermentative and/or methanogenic microorganisms.

The sludge may originate from the treatment of waste water, e.g., municipal or industrial waste water, or animal waste (e.g., manure). The sludge may be the residual sludge left after anaerobic and/or aerobic digestion of raw sludge. Thus, the sludge may contain high levels of heavy metals such as Zn, Pb, Cu, Cr, Ni, Cd, and Hg, and may contain high numbers of pathogenic organisms, including various enteric pathogens. The physical and/or chemical character of such sludge is not generally considered suitable for microbial processing.

The sludge processed by bioleaching is fed, or circulates to, one or more bioreactors for the biosynthesis of bioenergy products including, for example, methane, ethanol, butanol, and methanol, which are recovered and/or purified. For example, ethanol, butanol, or methanol may be produced by anaerobic fermentation of the organic material by one or more microorganisms such as certain bacteria, yeasts, and filamentous fungi, and the biofuel products are subsequently recovered. Alternatively, or in addition, methane may be produced by one or more methanogenic microorganisms (such as a microorganism consortium), and recovered and/or purified. Unprocessed organic material is subjected to further treatment, for example, by feedback through the system.

CO₂, as will be produced during anaerobic processes, may be used as a carbon source to support the growth and metabolism of photosynthetic microorganisms (e.g., blue-green algae) to synthesize additional biofuels, such as biodiesel (or synthetic intermediates such as lipids) and hydrogen gas.

In a second aspect, the present invention provides systems for generating biofuels and bioenergy products, for example, in accordance with the methods described herein. The system may comprises a bioleaching system, and a separate biosynthesis system. For example, treated sludge may be produced in a bioleaching system, and the resulting biomass (treated sludge) subsequently fed to the biosynthesis system. Such systems allow for the transportation of sludge treated at one location, to be transported to another for synthesizing biofuel or bioenergy products. Alternatively, the system may be an integrated system for coupling the bioleaching process with the biosynthesis of bioenergy products. The system may be connected to, or positioned or located near, the production or source of sludge, so as to obviate the need to transport the waste material for treatment or disposal.

The system comprises one or more bioreactors suitable for leaching heavy metals in the sludge material by the action of acid-producing, sulfur-oxidizing bacteria, to produce treated or processed sludge. For example, the bioleaching system may comprise at least one continuous stirred-tank reactor, and/or at least one tubular reactor, such as a recirculating tubular reactor or a tubular plug flow reactor. In some embodiments, the bioleaching system comprises a continuous stirred-tank reactor followed by a tubular plug flow reactor, thereby providing the aeration and mixing necessary to support industrial scale metabolism of sulfur-oxidizing bacteria. The system may further comprise a centrifuge for separating liquid and solid phases of sludge, such that solubilized metals may be precipitated from the liquid phase, and optionally recovered, with the solid phase used as a carbon source for biofuel production.

The system further comprises bioreactor(s) suitable for the biosynthesis of biofuels from the biomass. The bioreactor(s) for biosynthesis contain naturally selected or genetically engineered microorganisms for the synthesis of products such as, for example, ethanol, methanol, butanol, and methane from treated sludge. The biosynthesis reactor(s) may be anaerobic multiphasic bioreactor(s) having fermentative and/or methanogenic microbes forming biofilms on solid surfaces.

In certain embodiments, the biosynthesis system further comprises at least one photo bioreactor to support the production of additional biofuel products by photosynthetic microorganisms, including one or a consortium of algae(s). The metabolism of the photosynthetic microorganisms is supported by the CO₂ produced during anaerobic biosynthesis.

The system may further comprise mechanism(s) for collecting and/or recovering bioenergy products resulting from the biosynthesis processes. The system may further comprise a container or feed to recover non-fuel compounds for feedback to the bioleaching system. Thus, the system may comprise a feedback connection between the biosynthesis system and the bioleaching system to continuously recycle all materials not completely used, to avoid the production of pollutants.

Thus, in accordance with the invention, the hazardous disposal including incineration or damping of excess sewage sludge, as well as improper use as fertilizer, is avoided, while producing valuable biofuels including ethanol, butanol, and biodiesel among others.

### DESCRIPTION OF THE FIGURES

FIG. 1 is a flow diagram illustrating the method and system of the invention. Industrial, municipal, and/or farm sludge is treated in a leaching bioreactor to remove, for example, heavy metals from the sewage sludge. The treated sludge is used as a carbon source in a multiphasic bioreactor to produce biofuels. Remaining material is fed back through the system for further treatment and biosynthesis.

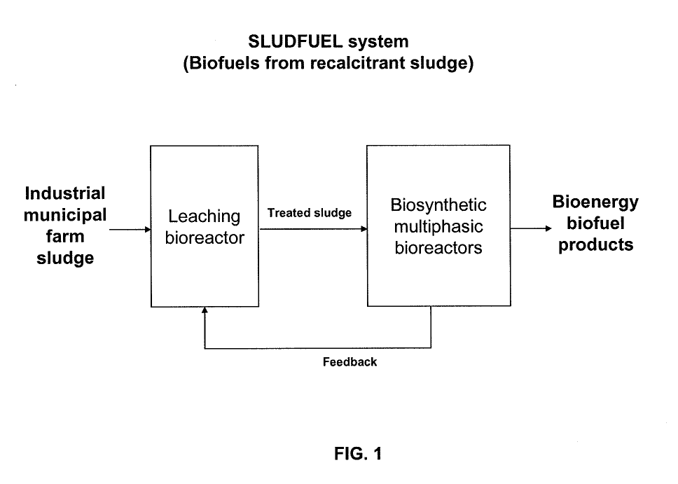

FIG. 2 illustrates an exemplary integrated system and process of the invention. The bioleaching reactor comprises an aerobic bioreactor working independently or connected in-series to the biosynthetic reactors. Treated sludge (heavy metals extracted) is fed to one or more bioreactors for synthesis of biofuels and bioenergy products. The multiphasic bioreactors for biosynthesis may comprise anaerobic bioreactor(s) and photo reactor(s). The bioleaching and the biosynthesis processes/systems are connected by a feedback loop, such that untreated waste and metabolites are recycled through the system.

FIG. 3 illustrates each bioreactor in the exemplary integrated system. The biosynthesis reactors contain a multiphasic fluidized bed to reach high conversion rates to product. A photosynthesis bioreactor is supported by the CO₂ emission from the anaerobic fermentation reactor.

FIG. 4 illustrates the structure of the fluidized biofilm formed in the multiphasic bioreactors. The multiphasic bioreactors each have solid or liquid surfaces, such as porous glass, silicone rubber, silicone oil, among others, to support microbial biofilms. Such multiphasic bioreactors maximize surface areas to support extensive microbial metabolism. The multiphasic bioreactors may contain, in addition to solid support surfaces, liquid surfaces as well as cellular, aqueous, and gas phases.

FIG. 5 shows the performance of a bioleaching reactor to remove heavy metals from sewage sludge. After 15 days of bioleaching the pH reached 2.3, and the solubilization of Zn, Cu, Pb and Cd reached (respectively) 79%, 81%, 65% and 60%. In contrast, metal solubilization in a control system (without solubilization source, e.g., sulfur substrate) was in the range of 3-6% for each metal.

FIG. 6 shows the performance of a biosynthetic multiphasic anaerobic reactor in the production of methane from treated sewage sludge. At the hydraulic retention time of 16 hours, approximately 70% and 46% of the chemical oxygen demand (COD) was converted to biogas with treated and untreated sludge respectively. About 80% of the COD was converted to biogas with ethanol as a carbon source.

FIG. 7 shows the performance of a biosynthetic multiphasic anaerobic reactor in the production of ethanol from treated sewage sludge. After 48 hours of culture, approximately 30% and 20% of COD was converted to ethanol using the treated and untreated sludge as a carbon source (respectively). As a control, approximately 50% of COD was converted to ethanol using glucose as a carbon source.

FIG. 8 shows the performance of a biosynthetic multiphasic photosynthetic reactor in the production of biodiesel from CO₂ outlet from an anaerobic bioreactor. Performance was assessed by the yields of biomass according to the concentration of CO₂ injected. Substantial biomass is achieved at 1% and 0.5% CO₂ concentrations, with more biomass produced at 1%. The lipids extracted to produce biodiesel reached approximately 65% of the biomass produced at different concentration of CO₂.

FIG. 9 shows the performance of a multiphasic photosynthetic bioreactor in the production of hydrogen in relation to biomass (nmol hydrogen per gram of protein). The hydrogen release was approximately 76 nmol of hydrogen per gram of protein when biomass was grown at 1% of CO₂, while 32 nmol of hydrogen per gram of protein was produced when 0.5% CO₂ was injected.

### DETAILED DESCRIPTION OF THE INVENTION

The present invention provides methods and systems for producing biofuel and bioenergy products using, as starting raw material, municipal, industrial, and/or farm sewage sludge, including recalcitrant sludge produced after anaerobic and/or aerobic digestion of raw sludge. In accordance with the invention, municipal, industrial, and farm sewage sludge, including recalcitrant sludge, can serve as a carbon source to support the metabolism of synthetic microorganisms to produce biofuels and bioenergy products.

### Sludge Materials

The present invention provides methods and systems for producing biofuel and bioenergy products using, as starting raw material, municipal, industrial, and/or farm sewage sludge or waste. The sludge may be treated municipal or industrial raw sludge or primary solids, or treated farm sludge. That is, the sludge in some embodiments has undergone one or more treatment processes such as anaerobic and/or aerobic digestion, composting, and/or at least one chemical or physical processing such as drying, dewatering, thickening, pressing, filtering, centrifugation, ultraviolet or chemical disinfection (e.g., chlorine disinfection), lime stabilization, and/or thermal processing.

For example, fresh sewage or wastewater may be treated in a settling tank, where approximately 50% of the suspended solid matter will settle out. This collection of solids is known as “raw sludge” or primary solids and is said to be “fresh” before anaerobic processes become active. Raw sludge may be passed to one or more digestion chambers where it is decomposed by anaerobic bacteria, resulting in liquefaction and reduced volume of the sludge. After digesting for an extended period, the resulting product is “digested” sludge, which can be a recalcitrant product to further bio-processing, and which is often disposed of by drying and then landfilling. This digested sludge, or “recalcitrant sludge,” may be used for the production of biofuels and bioenergy products in accordance with the invention.

Thus, in some embodiments, the sludge is a recalcitrant sludge having a high content of heavy metals such as one or more of Zn, Pb, Cu, Cr, Ni, Cd, and Hg. At least one, two, or three such heavy metal(s) (or heavy metals taken collectively) may be present at more than 10 ppm, or 50 ppm, or 100 ppm, or 200 ppm, or 400 ppm, or 500 ppm in the recalcitrant sludge. At least one such heavy metal (or heavy metals taken collectively) may be present at from about 400 to about 1000 ppm. For example, the recalcitrant sludge may be contaminated at such levels with lead (Pb) and/or cadmium (Cd).

In these or other embodiments, the sludge has a high content of at least one bacterial, viral, and/or parasitic pathogen, including a variety of enteric pathogens, for example, enteropathogenic E. coli, Salmonella, Shigella, Yersinia, Vibrio Cholerae, Cryptosporidium, Giardia, Entamoeba, Norovirus, and Rotavirus, among others.

Such recalcitrant sludge is not generally considered useful, and generally is a product ultimately applied in some manner to the environment, and having a deleterious effect to the soil, air, and/or public health. In accordance with the present invention, recalcitrant sludge is converted to one or more biofuel or bioenergy products thereby supplying needed energy while avoiding the negative impacts on the environment and public health caused by, for example, incineration of such recalcitrant sludge or its improper use for soil fertilization.

In certain embodiments, the sludge is generated by industrial activities. That is, the sludge is an end product of a waste water treatment plant of an industrial facility, such as a chemical, pharmaceutical, or paper production facility, or food processing facility.

For example, the sludge may originate from a pharmaceutical production facility. Most pharmaceuticals or their metabolites are excreted into urban wastewaters and eventually make their way to the municipal wastewater treatment. The removal of these substances through the municipal systems depends not only on the profile of the wastewater treatment (conventional activated sludge, biofilter, nitrification-denitrification systems) but also on the physico-chemical properties of the compounds. Pharmaceutical products that are not readily biodegradable during municipal treatment enter in the receiving waters as dissolved pollutants via municipal discharges or in agricultural fields via digested sludge. Effects of some pharmaceuticals, e.g. endocrine disrupters and antibiotics, in the aquatic environment are well documented, but not much is known about the behavior of these compounds in soils. Intake by plants, leaching into the groundwater and having negative impact on terrestrial organisms are possible. In accordance with the invention, such compounds may be degraded by microbial metabolism, either during the leaching or biosynthesis process, and converted to biofuels. Further, the combined metabolisms of the aerobic and anaerobic microorganisms (through cycling between leaching and biosynthesis systems as described herein), as well as the low pH of the bioleaching system, in some embodiments, promote the degradation of recalcitrant compounds.

In other embodiments, the sludge originates from a paper production facility. The pulp and paper industry is responsible for large discharges of highly polluted effluents. These pollutants, whose main characteristics are their high toxicity and low biodegradability, include a variety of tannins, lignins, resins, terpens, and chlorophenolic compounds. The composition of these effluents, which has a great influence on its treatability, may vary considerably, depending on the raw material and manufacturing process. The present invention, however, provides methods and systems that are versatile with regard to synthesizing biofuel components from these toxic effluents.

In still other embodiments, the sludge is generated by a food processing facility. Food processing wastewater, which due to its high COD-loading rates, and due to the content of some toxic compounds used during processing, must be decontaminated before being discharged in municipal systems.

In these or other embodiments, the sludge is an industrial sludge containing one or more chemical pollutants or contaminants, including recalcitrant xenobiotics. Such pollutants and contaminates may include one or more of a polychlorinated biphenyl (PCB), polycyclic aromatic hydrocarbon (PAH), dioxin, pesticide, endocrine disrupter, alkyl-sulfonate, alkylphenol, oil, grease, heavy metals, ammonia, or other aliphatic or aromatic hydrocarbon. Industries that generate wastewater with high concentrations of such pollutants often need specialized treatment systems, since sludge from such sources should not be applied directly to the environment and may not be suitable for municipal wastewater treatment. Such sludge may be converted to one or more biofuel and/or bioenergy products in accordance with the invention, while avoiding the impact of the contaminants on the environment, as well as avoiding the large costs of many specialized treatment systems.

In some embodiments, the sludge is farm sludge comprising animal manure. Recent growth of the livestock industries has given rise to new requirements for safe disposal of large quantities of animal waste (manure) generated at dairy, swine, and poultry farms.

Most animal manure is feces. Common forms of animal manure include FYM (farmyard manure) or farm slurry (liquid manure). Farmyard manure may also comprise plant material (often straw) which has been used as bedding for animals and has absorbed the feces and urine. Agricultural manure in liquid form is known as slurry, and is produced by more intensive livestock rearing systems where concrete or slats are used, instead of straw bedding.

The content of heavy metals in manure is a great concern when manure is used as a soil fertilizer. A comparison of heavy metal load in manure with that in other fertilizers shows that manure is responsible for about two thirds of the Cu and Zn load in fertilizers and for about 20% of the Cd and Pb load. The potential negative effects of heavy metals of swine, cattle and poultry slurry used to fertilize soils have been demonstrated. For example, pig slurry contains high concentrations of heavy metals and is significant hazard when applied directly as a solid fertilizer.

Thus, in accordance with the invention, the sludge may comprise animal manure originating from one or more of horse, cattle, pig, sheep, and/or poultry, among others. Manure from different animals may have different qualities, including varying levels of heavy metals preventing extensive bio-processing of the manure, and limiting its use as a fertilizer.

### Basic Media

Generally, the municipal, industrial, and/or farm sewage sludge (as described above) will contain organic and inorganic nutrients for supporting microbial growth and metabolism, e.g., in an aqueous phase. However, where these inorganic nutrients are absent, or are present in insufficient amounts, the sludge-containing material may be supplemented with an aqueous phase containing a basic mineral salt medium to support microbial growth and metabolism of sludge-digesting or sludge-treating microorganisms.

Thus, in the bioreactors (described more fully below), the aqueous phase(s) comprise inorganic nutrients to support microbial growth and metabolism. For example, the aqueous phase may comprise a mineral salts medium. Nitrogen and phosphorous are the main nutrients added to the aqueous phase. Micronutrients such as Ca, Zn, Mn, Cu, Fe, Mg, Mn, Mb, and S may also be present in at least trace amounts. An exemplary mineral salts medium is KHCO₃ (e.g., 2 g/L), NaHCO₃ (e.g., 1.8 g/L), KH₂PO₄ (e.g., 0.7 g/L), Na₂HPO₄.12H₂O (1.4 g/L), MgSO₄.7H₂O (e.g., 0.2 g/L), and (NH₄)₂SO₄ (e.g., 0.8 g/L). The medium may further contain trace elements as may be required to support growth and vitality of the microorganisms, such as Ca(H₂PO₄) (e.g., 40 mg/L), ZnSO₄.7H₂O (5 mg/L), Na₂MoO₄.2H₂O (2.5 mg/L), FeSO₄.7H₂O (1 mg/L), MnSO₄.H₂O (1 mg/L), and CuSO₄ (0.6 mg/L). The nutrient medium may of course be adjusted based upon the metabolic requirements of the microorganism(s) present for metal leaching and biosynthesis.

Generally, to support the leaching action of the acid-producing, sulfur-oxidizing bacteria, the presence of a sulfur substrate (e.g., sulfate) is necessary. The substrate may be present in the sludge material, or may be added in a basic media as described. For example, the substrate may be FeSO₄ at concentrations in the range of from about 2 to 40 grams per liter, or in the range of about 5 to 10 grams per liter.

### Bioreactors and Bioprocesses

The methods and systems of the invention employ a sludge treatment and processing system, and which in some embodiments is suitable for treatment and processing of recalcitrant sludges. The methods and systems employ one or more bioleaching reactors, and a biofuel synthesis system, which comprises one or more anaerobic fermentation and/or methanogenic bioreactors and optionally one or more photosynthetic bioreactors.

The invention involves feeding the sludge material (as described above) into a bioreactor system. The system generally comprises one or more reactors for bioleaching that will produce (e.g., in batch, semi-continuously, or continuously) a treated sludge material reduced significantly in concentrations of heavy metals and viable pathogens, and sufficiently processed/standardized for anaerobic fermentation or methanogenesis. The system further comprises one or more bioreactors for fermentation or methanogenesis of the organic material contained in the treated sludge, and in some embodiments a photosynthetic bioreactor operating off of the CO₂ effluent as a carbon source. The bioleaching and biosynthesis bioreactors may operate independently or be coupled to provide an integrated system.

The bioleaching reactor solubilizes metals by the acid produced by sulfur-oxidizing microorganisms. This process further results in the destruction of pathogenic microorganisms by low pH, as well standardization of the sludge material by degrading/dissolving the biosolids, making the resultant material suitable for anaerobic fermentation and/or methanogenesis. The bioleaching process may take place in high volumes (e.g., an industrial scale) as described in greater detail herein.

The type of bioreactors and internal designs may be selected on the basis of, for example, desired volume and/or retention time, as well as the microbial oxygen demand, and the desired flow and agitation systems.

For the bioleaching processes to be efficient, and to maximize the leaching kinetics within a very large volume (e.g., at an industrial scale as described herein), an important parameter is the supply of sufficient oxygen throughout the heap to support the oxidation reactions. Thus, the reactor design should be sufficient to provide sufficient mixing with sufficient oxygenation. For example, the bioleaching system may comprise at least one continuous stirred-tank reactor, or a similar reactor design with a sufficient aeration mechanism. Alternatively, or in addition, the leaching system may comprise a tubular recirculating reactor, or an air-lift reactor, or a rotary reactor.

In some embodiments where heavy metals are present in high concentrations, such as from about 3 to about 6 g/L (or as described previously), the bioleaching system may comprise a continuous stirred tank reactor followed by a tubular reactor, such as a plug flow reactor. Because of the need for aeration and due to the presence of solid particles/mass, the performance of the leaching system is enhanced in some embodiments with at least one long tubular reactor.

The bioleaching reactor may contain a mechanism for injection of oxygen or an oxygen source, to support high levels of sulfur oxidation.

The bioleaching reactor employs the action of acid-producing bacteria, which may be indigenous to the sludge material, to solubilize metals and reduce pathogen counts. Metal solubilization and pathogen destruction is accomplished by obtaining a pH in the system in the range of from 1 to 4, or from 1 to 3.

The sludge or the liquid phase containing solubilized metals may flow from the bioleaching reactor to a recipient system or unit where the biomass is recovered. For example, the recipient system or unit may comprise a centrifuge for separating biomass from the liquid phase containing solubilized heavy metals. The solubilized metals may then be removed and/or recovered, for example, by precipitation from the liquid phase. Precipitation of heavy metals can be accomplished by restoring the pH of the liquid phase to the neutral, near neutral, or basic pH range. The aqueous phase having its pH restored to neutral, near neutral, or basic range (and heavy metals removed), may be added back to the biomass to provide the requisite pH for biofuel production. Precipitated metals may be recovered and recycled for other uses.

Once the sludge material is treated by the bioleaching system, e.g., significant amounts of heavy metals have been removed, and pathogen counts reduced or entirely eliminated, the treated sludge (or biomass) having its pH restored to neutral or near neutral range (e.g., in the range of 5 to 7.5, or in the range of 6 to 7, or to about 6.5) is transferred or flows to a biosynthesis reactor to produce biofuels. Generally, the biosynthesis system will comprise an anaerobic bioreactor for fermentation or methanogenesis of the organic material. For example, the anaerobic bioreactor may be a UASB methanogenic (methane-producing) digester or an expanded granular sludge bed (EGSB) digester.

An Upflow Anaerobic Sludge Blanket (USAB) reactor uses an anaerobic process whilst forming a blanket of granular sludge which suspends in the tank. The treated sludge flows upwards through the blanket and is processed by the anaerobic microorganisms. The upward flow combined with the settling action of gravity suspends the blanket with the aid of flocculants. Small sludge granules begin to form whose surface area is covered in aggregations of bacteria. In embodiments having an absence of a support matrix, the flow conditions create a selective environment in which only those microorganisms, capable of attaching to each other, survive and proliferate. Eventually the aggregates form into dense compact biofilms referred to as “granules.”

The UASB reactor is a high rate anaerobic system for sewage treatment and methane production. However, conventionally, the sludge became concentrated with high fractions of heavy metals. In such conditions the performance of the UASB reactors has been limited. Thus, the invention provides an integrated or independent bioleaching system, as described, in connection with a UASB reactor, to support industrial scale synthesis of biofuels from sludge, including even recalcitrant sludge. The heavy metals which typically hinder sludge digestion in a UASB-type reactor, are removed in the bioleaching reactor, such that the treated sludge is efficiently digested in a fluidized UASB reactor.

An Expanded Sludge Bed Digester (EGSB) reactor is a variant of the UASB-type reactor. The EGSB reactor allows for a faster rate of upward-flow for the wastewater passing through the sludge bed. The increased flux permits partial expansion (fluidization) of the granular sludge bed, improving wastewater-sludge contact as well as enhancing segregation of small inactive suspended particle from the sludge bed. The increased flow velocity is either accomplished by utilizing tall reactors, or by incorporating an effluent recycle (or both).

Ethanol production may be performed in a fluidized bed recirculating tubular bioreactor containing a yeast (e.g., Saccharomyces sp.) or bacteria (Zymomonas sp.) attached and forming biofilms to suspended solid or liquid supports. The ethanol reactor may work independently, or may operate in series to receive biomass from the bioleaching system, and to support one or more photosynthetic bioreactors by the effluent CO₂ from the anaerobic process.

The biofuel synthesis system may also comprise at least one photosynthesis bioreactor, which may be a multiphasic system containing photosynthetic microorganisms such as blue green algae and other microalgae species described herein, which may be supported by effluent CO₂ from the anaerobic bioreactors. Photo reactors are known, such as those described in U.S. Pat. No. 7,371,560, which is hereby incorporated by reference in its entirety. The aeration, temperature, pH, nutritional requirements, intensity and wavelength of light (e.g., white light), as well as duration of light/dark cycles suitable for photosynthetic microorganisms are known, for example, as also described in U.S. Pat. No. 7,371,560.

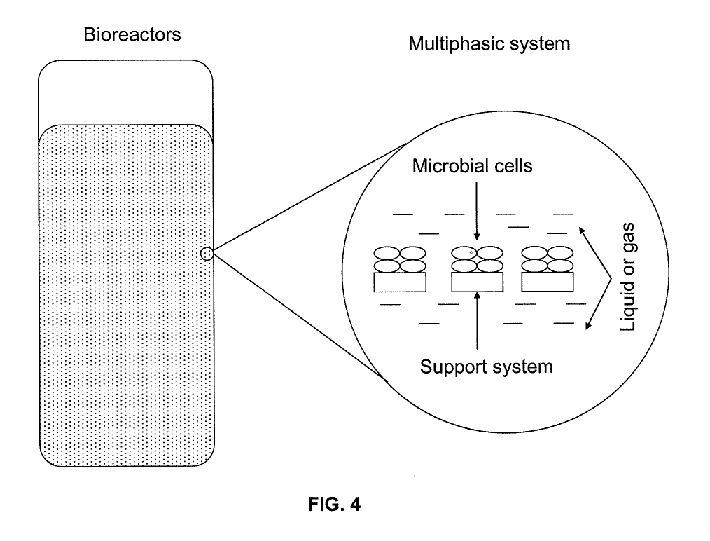

In some embodiments, the anaerobic bioreactors for synthesis of biofuels and bioenergy products may harness microbes in aqueous suspension and/or supported on surfaces. For example, the bioreactors may comprise solid surfaces that support biofuel-synthesizing microbes within biofilms. The solid surfaces may be composed of a variety of materials including porous glass, silicone rubber, as well as polymeric or metal surfaces (for example). The solid surfaces may form a fixed-bed reactor, that is, via a fixed solid support matrix (see FIG. 4). Alternatively or in addition, one or more support surfaces may be in the form of polymeric beads and the like, which may form a support bed or support matrix.

In some embodiments, the bioreactors for biosynthesis are multiphasic reactors having solid phases (support surfaces and microbial cells), liquid phases (aqueous and/or organic phases), and gas phases (air, and gas produced by microbial metabolism). Aqueous liquid and gas phases may circulate within or between the various reactors as described herein. Where aqueous and organic (oil) phases are employed, microbes may form biofilms at the liquid interface as well as on the solid support surfaces.

### Microorganisms

Microorganisms suitable for leaching heavy metals, as well as for biofuel and bioenergy product synthesis, are known. Exemplary microorganisms are listed in Tables 1 and 2 (below).

Exemplary microorganisms known to have metal leaching activity are provided below (Table 1), together with exemplary metals and enzymes involved. Such microorganisms are generally acid-producing, sulfur-oxidizing bacteria, and may be indigenous in sewage sludge. The activity of these microbes may be activated in the sludge by the presence of sulfur substrate and sufficient oxygen to support the oxidation reactions.

The main enzymes involved in metal solubilization are the iron oxidase, hydrogen sulfide:ferric ion oxidoreductase (SFORase), and cytochrome c oxidase. Examples of enzymes for leaching heavy metals are listed below in Table 1. These enzymes may be genetically engineered and introduced into suitable host microbes to increase the enzymatic performance.

TABLE 1
Microorganisms and Enzymes For Extraction
of Heavy Metals by Bioleaching
Sewage sludge

| | |
|---|---|
| Exemplary | Lead |
| heavy metals | Zinc |
| | Copper |
| | Cadmium |
| | Mercury |
| | Iron |
| | Nickel |
| | Chromium |
| Enzymes | iron oxidase |
| involved | hydrogen sulfide: ferric ion oxidoreductase (SFORase) |
| | cytochrome c oxidase |
| Organisms | Acidithiobacillus thiooxidans |
| | Leptospirillum ferrooxidans |
| | Acidithiobacillus ferrooxidans |
| | Thiobacillus thiooxidans |
| | Thiobacillus ferrooxidans |
| | Acidiphilium cryptum |
| | Acidiphilium multivorum |
| | Acidiphilium symbioticum |
| | Acidiphilium angustum |
| | Acidocella aminolytica |
| | Acidocella facilis |
| | Sulfobacillus thermosulfidooxidans |
| | Ferroplasma acidarmanus |
| | Metallosphaera sedula |
| | Sulfolobus acidocaldarius |
| | Sulfolobus solfataricus |

The microorganisms for biofuel synthesis may, for example, include one or a consortium of bacteria as methanogens, yeasts such as Saccharomyces sp., anaerobic bacteria such as Clostridium sp., or microalgae such as Chlorella sp. or Synechococcus sp., among others. Exemplary microorganisms having known biosynthesis activity are provided below (Table 2), together with exemplary enzymes involved in biosynthesis pathways. Examples of enzymes for synthesizing particular biofuel compounds are also provided. These enzymes may be genetically engineered to increase their performance.

Where methane is a desired biofuel product, at least one bioreactor for biosynthesis is an anaerobic reactor that comprises a methanogenic microorganism, which may include one or a consortium of Methanobacterium sp., Methanothrix sp., Methanosarcina sp., and Methanomonas sp. Other methanogenic microbes that may be used, and are described in U.S. Pat. No. 6,555,350, which is hereby incorporated by reference. For example, methanogens also include Methanococcus sp, Methanomicrobium sp., Methanospirilliam sp., Methanoplanus sp., Methanosphaera sp., Methanolobus sp., Methanoculleus sp., Methanosaeta sp., Methanopyrus sp., and/or Methanocorpusculum sp.

TABLE 2
Microorganisms and Enzymes for Biosynthesis of Biofuels and Bioenergy Products
Methane

| | |
|---|---|
| Exemplary | Organic acids, CO2 |
| carbon sources | |
| Enzymes | formylmethanofuran dehydrogenase, |
| involved | methyltetrahydro-methanopterin: coenzyme M methyltransferase (Mtr), |
| | heterodisulfide reductase (Hdr), |
| | F₄₂₀H₂ oxidase (FprA), |
| | formaldehyde activating enzyme (Fae) |
| | methenyltetrahydromethanopterin cyclohydrolase, |
| | methylenetetrahydromethanopterin reductase, |
| Exemplary | Methanococcus sp, |
| Organisms | Methanomicrobium sp. |
| | Methanospirilliam sp. |
| | Methanoplanus sp. |
| | Methanosphaera sp. |
| | Methanolobus sp. |
| | Methanoculleus sp. |
| | Methanosaeta sp. |
| | Methanopyrus sp. |
| | Methanocorpusculum sp. |
| | Methanosarcina |

Ethanol

| | |
|---|---|
| Exemplary | Biomass, and carbonic metabolites produced after xenobiotic biodegradation |
| carbon sources | |
| Enzymes | Alcohol dehydrogenase (A, B, and C) |
| involved | Acetaldehyde dehydrogenase |
| | Amylases |
| | Glucoamylases |
| | Invertases |
| | Lactases |
| | Cellulases |
| | Hemicellulases |
| Exemplary | Saccharomyces sp. |
| Organisms | Klyveromyces sp. |
| | Zymomonas sp. |

Butanol

| | |
|---|---|
| Exemplary | Biomass, and carbonic metabolites produced after xenobiotic biodegradation |
| carbon sources | |
| Enzymes | Acetyl-CoAacetyltransferase |
| involved | Acetoacetyl-CoAthiolase |
| | 3-hydroxybutyryl-CoAdehydrogenase |
| | Crotonase |
| | Butyryl-CoAdehydrogenase |
| | Aldehyde/alcohol dehydrogenase |
| Exemplary | Clostridium sp. |
| Organisms | |

Hydrogen

| | |
|---|---|
| Exemplary | Water and light |
| sources | |
| Enzymes | Hydrogenases |
| involved | |
| Exemplary | Clostridium sp. |
| Organisms | |

Biodiesel

| | |
|---|---|
| Exemplary | CO2, and light |
| carbon source | |
| Enzymes | Lipasas (Triacylglycerolhydrolases) |
| involved | |
| Exemplary | Chlorella sp. |
| Organisms | Synechococcus sp. |
| | Synechocystis sp. |
| | Nitzchia sp. |
| | Schizochytriu sp. |

Methanol

| | |
|---|---|
| Exemplary | Methane and oxygen |
| carbon source | |
| Enzymes | Methane monooxygenase |
| involved | Formate dehydrogenase |
| | Formaldehyde dehydrogenase |
| Exemplary | Methylomonas sp. |
| Organisms | Methylosinus sp. |
| | Methylococcus sp. |

Some methanogenic species are highly thermophilic and thus can grow at temperatures in excess of 100° C. Where the methanogen is highly thermophilic, a separate (e.g., independent) bioreactor for synthesis of methane may be preferred. In certain embodiments, the methanogen is one or a consortium of Methanosarcina, Methanosaeta and/or Methanothrix species, which may carry out conversion of acetate and similar small-molecule carbon substrates to methane and carbon dioxide. Methanogens may use small organic compounds as substrate, such as formic acid (formate), methanol, methylamines, dimethyl sulfide, and methanethiol, which may be produced in the system.

The methanogenic bacteria may be naturally-selected for high methane producing activity, or alternatively, may be genetically modified by known techniques. The methanogenic enzymes that may be genetically engineered include, formylmethanofuran dehydrogenase, methyltetrahydro-methanopterin: coenzyme M methyltransferase (Mtr), heterodisulfide reductase (Hdr), F₄₂₀H₂ oxidase (FprA), formaldehyde activating enzyme (Fae), methenyltetrahydromethanopterin cyclohydrolase, and methylenetetrahydromethanopterin reductase. These enzymes have been isolated from species including, Methanococcus, Methanothermobacter, methanosarcina, methanopyrus, among others.

Biogas may be produced during anaerobic decomposition of the treated sludge. “Biogas” is a product of anaerobic digestion. In the absence of oxygen, anaerobic bacteria decompose organic matter and produce a gas mainly composed of methane (about 60%) and carbon dioxide. This gas can be compared to natural gas, which is approximately 99% methane. Biogas can be collected and used as an energy source for generators, boilers, burners, dryers or any equipment using propane, gas or diesel. Alternatively, methane may be recovered from the biogas as described below.

The process and systems described herein may be designed to produce alcohols, such as alcohols containing one to nine carbon atoms. Particular examples of alcohols that can be produced according to the invention include propanol, butanol, pentanol, hexanol, heptanol, octanol and nonanol. For the synthesis of alcohols, particularly ethanol, at least one bioreactor is an anaerobic reactor comprising fermentative microorganisms, such as one or more Zymomonas sp. and/or Saccharomyces sp. (e.g., Saccharomyces cerevisiae). Additional microorganisms suitable for fermentation of the biomass include a number of yeasts such as Klyveromyces sp., Candida sp., Pichia sp., Brettanomyces sp., and Hansenula sp. and Pachysolen sp. Alternatively, the microorganism may be one or a consortium of bacterial species such as Leuconostoc sp., Enterobacter sp., Klebsiella sp., Erwinia sp., Serratia sp., Lactobacillus sp., Lactococcus sp., Pediococcus sp., Clostridium sp., Acetobacter sp., Gluconobacter sp., Aspergillus sp., and Propionibactedum sp.

Various organisms for the production of fermentation products are known, as well as conditions for growth and substrate requirements, and are described for example, in U.S. Pat. No. 7,455,997, U.S. Pat. No. 7,351,559, U.S. Pat. No. 6,555,350, and U.S. Pat. No. 7,354,743, which descriptions are hereby incorporated by reference. In certain embodiments, the desired product is butanol, and the fermentative microorganisms include one or a consortium of bacteria including Clostridium sp.

The fermentative microorganisms, for example to produce ethanol, may be naturally selected for the production of the desired product, or may be genetically engineered to express desired enzymes. Techniques for genetic manipulation of bacteria and yeasts are well known, and include introduction of extrachromosomal elements by plasmid or phage, or integration of such elements into the host genome. Exemplary enzymes that may be genetically engineered include, Alcohol dehydrogenase (A, B, and C), Acetaldehyde dehydrogenase, Amylases, Glucoamylases, Invertases, Lactases, Cellulases, and Hemicellulases, among others.

In certain embodiments, the process and systems described herein will include a photo reactor to convert CO₂ produced during anaerobic biofuel synthesis to biofuel products such as hydrogen gas and lipids. Lipids may be employed in the production of biodiesels, for example, by transesterification. Photosynthetic organisms for use in the production of hydrogen gas and lipids from CO₂ are known, and include one or a consortium of naturally selected or genetically modified Synechococcus sp., Chlorella sp., Synechocystis sp., Nitzchia sp., and/or Schizochytriu sp., among others.

For the production of hydrogen, the method and system may employ photosynthetic microorganisms capable of using water as an indirect substrate for hydrogen production. Such microorganisms generally express one or more hydrogenases. These may include cyanobacteria and algae, such as green algae, blue-green algae, or red algae. Exemplary species include Synechococcus sp., Chlorococcales sp. and Volvocales sp., among others.

The aeration, temperature, pH, nutritional requirements, intensity and wavelength of light (e.g., white light from natural or artificial light source), as well as duration of light/dark cycles suitable to support the growth and metabolism of photosynthetic microorganisms are known, for example, and are described in U.S. Pat. No. 7,371,560 which is hereby incorporated by reference.

### Operating Conditions

The bioreactor(s) are inoculated, where necessary, with the selected microorganism or mixed culture, followed by an acclimation period. The acclimation period may last one month, two weeks, one week, or less, during which biofilms, where present, are formed on support surfaces and the desired metabolic processes induced. Acclimation may involve, for example, induction or derepression of enzymes, multiplication of the initially small population(s) of bioleaching or synthetic microorganisms, selection for beneficial mutations, optimization of inorganic nutrients or other conditions, adaptation of microorganisms to toxins or inhibitors that may be present, and predation by certain microorganisms (e.g., protozoa).

Acclimation in some embodiments may proceed in steps, by first activating bioleaching bacteria for input sludge material, followed by acclimating biosynthetic microorganisms for the production of the desired product(s). When using an integrated system as described herein, the flow of sludge material from the bioleaching reactors to the anaerobic biosynthesis reactor can be controlled or initiated once bioleaching bacteria are fully or sufficiently induced or acclimated. Such embodiments may be useful particularly where the sludge material contains components that are toxic to biosynthetic microbes.

During the induction/acclimation period, it may be important to limit the concentration of the sludge material and/or the flow of sludge through the system. Growth and selection of bioleaching microorganisms may be evaluated on the basis of the pH, or changes in concentrations of solubilized heavy metals. Growth and selection of biofuel-synthesizing microorganisms may be evaluated by the appearance and concentration of product being produced, as well as by the production of the expected metabolites (e.g., CO₂, methane, ethanol, butanol, hydrogen, etc.).

During acclimation and after acclimation, the bioreactor conditions may be adjusted as necessary to optimize product yield and rate of synthesis. Such conditions include sludge input concentration, flow rate, bioreactor temperature(s), levels of bioreactor agitation, pH, and levels of aeration or oxygenation. For example, the temperature of bioleaching and anaerobic reactors may be maintained within the range of about 15° C. to about 35° C., such as about 18 to about 32° C. The flow-rate of substrate through the system may depend on the volume of the bioreactor, and the bioleaching and biosynthesis rates, and may be maintained by a system of pumps and/or valves. The bioreactor may further allow for agitation of the liquid material, if necessary to maintain the availability of nutrients.

After or continuously during the leaching process, the biomass and liquid phase will be separated (e.g., by centrifugation), and heavy metals precipitated from the liquid phase by restoring the pH. The precipitated heavy metals may thereafter be removed (e.g., by centrifugation). For example, upon efficient leaching (metal solubilization), the pH of the sludge will be in the range of from about 1 to 3. The metals are thus removed and/or recovered from the liquid phase by restoring the pH to near neutral or higher to precipitate the metals from solution. With its pH restored and heavy metal precipitate removed, the liquid phase can be used to restore the pH of the biomass before biofuel synthesis (e.g., anaerobic fermentation or methanogenesis). For example, the pH of the biomass will generally be restored to near neutral (e.g., in the range of 5.0 to 7.5, such as about 6.5). The pH of the liquid phase or biomass may be restored with sodium hydroxide or other suitable base.

In certain embodiments, complete bioleaching of the sludge may take place first, independently of biofuel synthesis, or alternatively, biosynthesis may take place simultaneously with bioleaching in a coupled bioleaching/synthesis bioreactor. In certain embodiments that employ independent bioleaching and biosynthesis reactors, the bioleaching process may proceed for from about 3 hours to about 1 week, or about 10 hours to about 3 days, or in certain embodiments, for about 1, about 2, about 3, about 4, or about 5 days. For example, bioleaching may proceed for (be substantially complete at) about 3 hours, about 5 hours, about 10 hours, about 15 hours, or about 24 hours. Alternatively, a coupled/integrated bioleaching and biosynthesis system may convert sludge to biofuel in about 1 week or less, about 4 days or less, about 2 days or less, about 1 day or less, about 15 hours or less, or about 10 hours or less. The length of time needed for the bioprocesses will depend on several conditions, including, sludge input concentration, flow rate, volume of bioreactors, bioreactor temperature(s), levels of bioreactor agitation, and levels of aeration or oxygenation.

In certain embodiments, biofuel or bioenergy production is at an industrial scale with a continuous or semi-continuous integrated bioleaching/biosynthesis system, such that from about 100 to about 100,000 gallons of sludge substrate are degraded per day. For example, about 500 to about 10,000 gallons of sludge substrate may be processed and converted to biofuel in a period of about 24 hours to about 48 hours. In certain embodiments, about 500 to about 10,000 gallons of sludge substrate may be processed and converted to biofuel in a period of less than about 24 hours.

### Recovery of Biofuels

Biofuel products may be recovered and/or purified by known and commercially available methods and devices.

Alcohols such as ethanol, methanol, and/or butanol may be recovered from liquid material by molecular sieves, distillation, and/or other separation techniques. For example, ethanol can be concentrated by fractional distillation to about 90% or about 95% by weight. There are several methods available to further purify ethanol beyond the limits of distillation, and these include drying (e.g., with calcium oxide or rocksalt), the addition of small quantities of benzene or cyclohexane, molecular sieve, membrane, or by pressure reduction.

Product gas, for example, as produced by anaerobic metabolism or photosynthesis, may be processed to separate the methane and/or hydrogen components. Methane, hydrogen, or biogas may be drawn off from the system as pipeline gas.

In accordance with the invention, methane and/or hydrogen may be recovered as a biofuel product. Methane may be recovered and/or purified from biogas by known methods and systems which are commercially available, including membrane systems known for separating gases on the basis of different permeabilities. See, for example, U.S. Pat. No. 6,601,543, which is hereby incorporated by reference. Alternatively, various methods of adsorption may be used for separating methane and hydrogen.

Other ways of collecting biofuel products including centrifugation, temperature fractionalization, chromatographic methods and electrophoretic methods.

In certain embodiments, the biofuel recovery/purification components may be integrated into the system, for example, by connecting the respective device or apparatus to the gas or liquid effluents from the biosynthetic bioreactors. The purified biofuels and bioenergy products may be stoked in a separate container(s).

### Integrated Systems

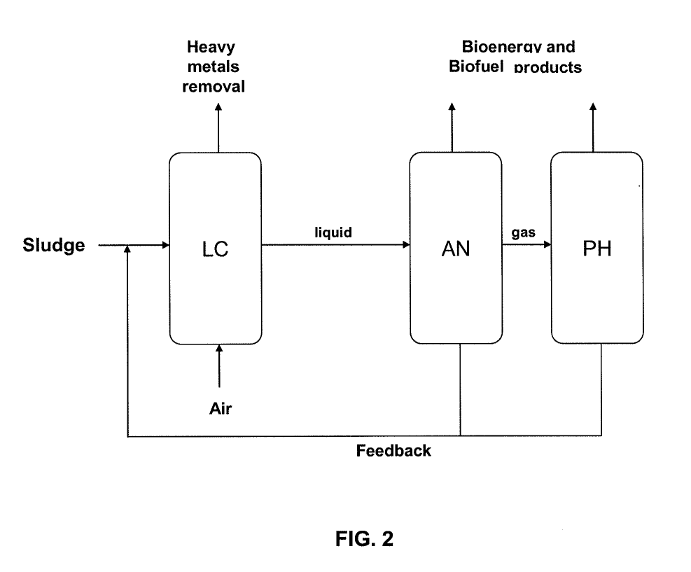

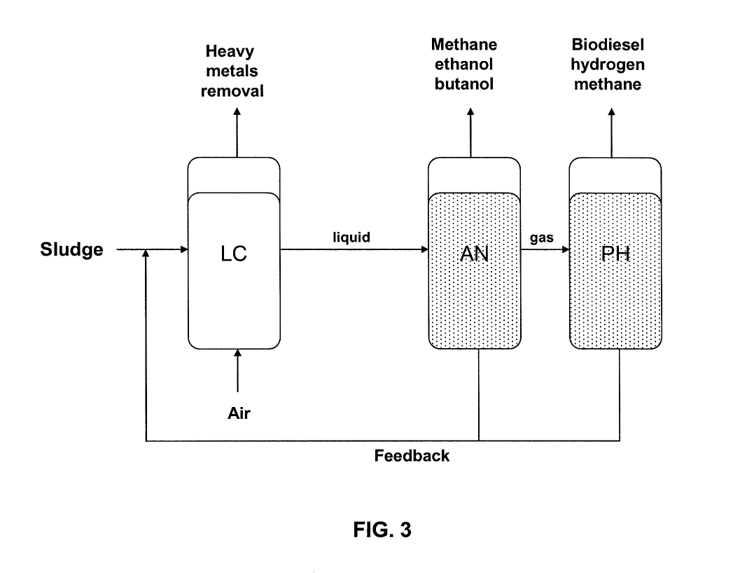

The present invention further provides an integrated in-series system for generating biofuels, such as hydrogen and methane, as well as other useful products such as ethanol, butanol, methanol, and biodiesel. Exemplary integrated systems are illustrated in FIG. 2 and FIG. 3.

The integrated system comprises one or more leaching systems or bioreactors suitable for removing the excess of heavy metals from sewage sludge. The leaching bioreactors, which contain the leaching bacteria in suspension with the sludge, contain an inlet for influent sewage sludge liquid (including recalcitrant sludge materials as described) to the bioleaching reactor system. In certain embodiments, the bioleaching reactor further contains a first outlet for effluent liquid containing solubilized heavy metals, so as to allow removal of these heavy metals, for example, in a recipient system or unit. In some embodiments, the bioleaching system further comprises a second outlet for effluent liquid having a reduced level of heavy metals as a result of the bioleaching treatment. Alternatively, the recipient system for removing heavy metals from the liquid phase may operate in series after the leaching process.

The bioleaching system may employ a continuous stirred-tank reactor and/or an in-series tubular reactor, such as a recirculating tubular reactor or a plug flow reactor as described. Alternatively, the bioleaching system may comprise an air-lift reactor or a rotary reactor.

The recipient system for removal of heavy metals from solution, comprises one or more centrifuges for separating the liquid phase from biomass, and a mechanism for siphoning off the liquid phase to restore the pH and remove precipitated heavy metals. The recipient system may further comprise a mechanism for adjusting the pH of the liquid phase and/or biomass, such as an inlet, sampling port, and/or pH meter.

The recipient system may operate in series, between the bioleaching reactor and anaerobic reactor, or may operate as an independent unit.

The integrated system further comprises an in-series anaerobic bioreactor, and optionally a photosynthesis bioreactor, to synthesize biofuels and bioenergy products from the treated sludge. The anaerobic bioreactor contains an inlet for treated sludge from the bioleaching system, and an in-series connection to transport gas effluent from the biosynthesis anaerobic reactor to an algae photo bioreactor. The algae photo reactor may contain an inlet for liquid media containing necessary salts and inorganic nutrients (e.g., sea water or artificial medium having a similar chemical makeup). The anaerobic bioreactor system further comprises an outlet from the anaerobic biosynthesis bioreactor transporting the biofuels and bioenergy products, and an outlet from the photo bioreactor to transport the biofuels and bioenergy products.

In some embodiments, the anaerobic bioreactor may be a UASB reactor or an expanded granular sludge bed (EGSB) reactor for production of methane.

In some embodiments, the anaerobic and photo bioreactors internally may contain liquid surfaces (silicone oils) and solid surfaces (porous glass, silicone rubber, etc), where growing microorganisms form biofilms. The microorganisms growing in the anaerobic and photo bioreactors are mixed cultures of naturally selected or genetically engineered cells as described.

The system further comprises a feedback connection between the biosynthesis system to the bioleaching reactor to recycle all materials not completely degraded or used, to obtain synergism between the aerobic and anaerobic microorganisms and/or pH changes within the system, and to obtain a virtual “zero pollution system.”

A pump system may be employed for connecting inlets and outlets, to and from bioreactors, to maintain the desired flow through the system. Valves may also be present to allow for the flow of material through the system to be controlled.

The system may include sampling ports, so that concentrations of heavy metals and/or biofuel production can be monitored, as well as oxygen consumption, CO₂ production, pH, oxidation-reduction conditions, microbial cell physiology, biofilm health, among others.

The integrated system is generally a continuous system, with material circulating between the bioleaching and biosynthesis reactors, as controlled by the system of pumps. For example, in the continuous system, the anaerobic biosynthesis reactor may operate with an incoming flow rate equal to the outlet flow rate of liquid from the recipient unit or the bioleaching reactor (e.g., the flow rate of liquid from the first and/or second outlets of the bioleaching reactor). Further, the photo reactor may operate with a gas influent that is equivalent to the effluent gas of the anaerobic reactors.

In certain embodiments, as illustrated in FIG. 3, the integrated system is a coupled bioleaching system and a multiphasic biosynthesis system. The multiphasic reactor system for biofuels and bioenergy production comprises a multiphasic anaerobic bioreactor for biosynthesis, and a multiphasic photo bioreactor for biosynthesis. Multiphasic bioreactors have been described (see FIG. 4).

The working volume of the system may be from about 100 gallons to about 100,000 gallons. For example, the industrial system may convert from about 500 to about 10,000 gallons of source material to useful product per run (in batch), or per day (when continuous). The operation or retention time for converting product to fuel may be about 2 weeks or less, but in some embodiments is 1 week or less, such as 3 days, 2 days, or 1 day or less (e.g., 15 hours).

In certain embodiments, the system is configured to allow for the transportation of treated sludge produced at one location (by a bioleaching reactor as described herein), to be transported to another location for synthesizing biofuel or bioenergy products (by a biosynthesis bioreactor as described herein). Alternatively, the system may be entirely integrated to couple bioleaching with the biosynthesis of bioenergy products from the treated material. The system (or the bioleaching reactor) may be connected to, or positioned or located near, the production or source of such sludge, so as to obviate the need to transport the waste for disposal. For example, the system or bioleaching components may be located within about 1 mile or less of the production or source of the sludge waste.

### EXAMPLES

### Example 1

### Testing of a Bioleaching Reactor for the Removal of Heavy Metals from Sewage Sludge

This example demonstrates that heavy metals from sewage sludge can be removed using a bioleaching reactor. The sewage sludge contained high levels of heavy metals including Pb, Cd, Cu, Hg, and Zn. When sludge is disposed as biosolids in soil, the heavy metals are accumulated in soil and can be transferred to the plant cultures.

An alternative to chemical processes for removing heavy metals is microbial leaching with certain bacteria species. Bioleaching in accordance with the invention has several advantages over chemical methods in terms of its simplicity, high yield of metal extraction, lower acid and alkali consumption, and minimum reduction in sludge nutrients such as N and P.

The activity of leaching bacteria can be enhanced with additive Fe(II) as the source substance for Thiobacillus sp. bacteria. The sulfur-oxidizing bacteria are naturally available in the sludge samples and can be activated by providing sulfur and aeration at about 28° C. to 30° C.

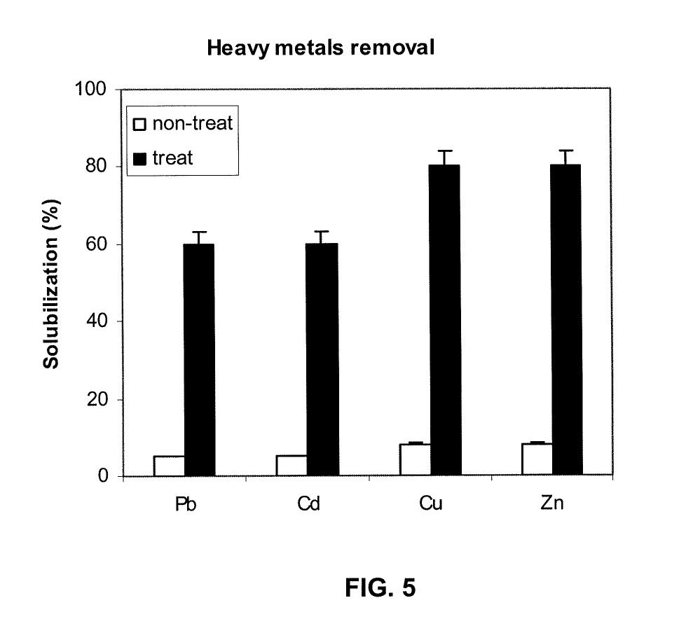

A mixed culture of indigenous Thiobacillus sp. were cultured in a 1-L reactor containing sewage sludge, FeSO₄, and sulphur powder as substrates to assess heavy metal bioleaching from the sewage sludge. The reactor was maintained at 30° C. with active oxygenation. Activation resulted in bio-acidification to pH 2 within 5-11 days. Successive inoculation of fresh sludge with 5% acidified samples reduced the acidification time to 2 to 3 days in most samples. As is shown in FIG. 5, after a 15-day bioleaching period the pH reached 2.33, and the solubilization of Zn, Cu, Pb and Cd reached, respectively, 79%, 81%, 65% and 60%. In contrast, metal solubilization in a control system (without sulfate source) was only around 3 to 6%. Compared with this control system, the addition of FeSO4 (5-10 g/l) accelerated the heavy metal solubilization and removal from sludge.

These results demonstrate that the bioleaching reactor was highly efficient in the removal of heavy metals, including Pb, Zn, Cu, and Cd. This process can be used efficiently at an industrial level to remove heavy metals from sewage sludge, to thereby condition the sludge for production of biofuels.

### Example 2

### Testing of a Multiphasic Bioreactor for the Production of Methane from Treated Sewage Sludge

This example shows that the treated (by bioleaching) sewage sludge can be efficiently digested to produce methane. The aim of bioleaching treatment is to improve or allow the anaerobic digestion of the sludge, where high concentrations of heavy metals otherwise make sludge recalcitrant to anaerobic digestion.

Batch tests were performed in a 1-L UASB anaerobic reactor to assess sludge biodegradability and production of methane. The reactor was fed with sludge previously treated to remove heavy metals by the bioleaching process. A bacteria consortium of a previous digested sludge was used as inoculums. Treated sludge, non-treated sludge, as well as a control sample consisting of ethanol, were digested. The organic matter was equivalent to 8 g COD/L in all samples.

The biogas volume produced was measured by movement of liquid (water, pH 2, NaCl 10%). Biodegradability was assessed by comparison of biogas volumes produced by treated, untreated and control samples. The biodegradable percentage was estimated by comparing the biogas volume produced with sludge (treated or untreated) to the biogas volume produced with control.

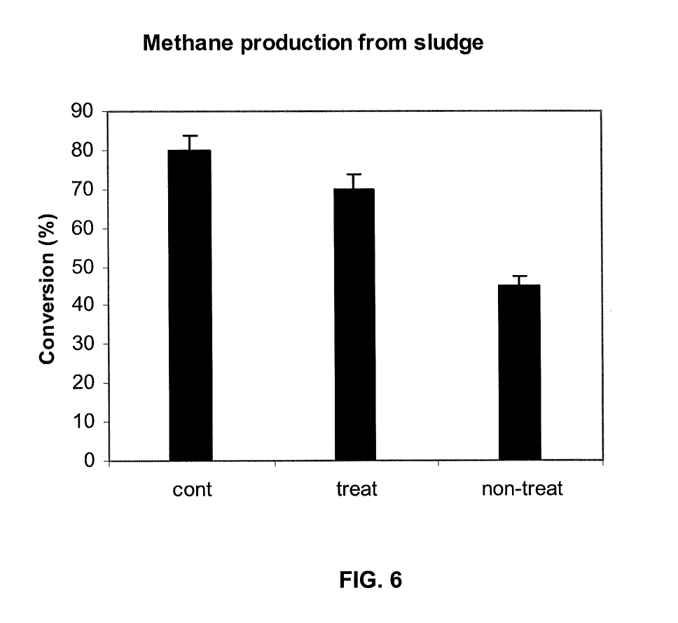

The results are shown in FIG. 6. At the hydraulic retention time of 16 hours, approximately 70% and 46% of the chemical oxygen demand (COD) was converted to biogas with treated and untreated sludge respectively. About 80% of the COD was converted to biogas with ethanol as a carbon source.

These results indicate that sludge treated to remove heavy metals allows higher biogas production than with untreated sludge, and close to an ethanol control. These results also demonstrate that the heavy metal removal increased the production of methane about 25% in relationship to the non-treated sludge.

### Example 3

### Testing of a Multiphasic Bioreactor System for the Production of Ethanol from Treated Sewage Sludge

This example demonstrates the production of ethanol from treated (heavy metals removed) sewage sludge.

A 1-L bioreactor containing 500 ml of sludge (8 g COD/L) was inoculated with a selected culture of the yeast of Saccharomyces sp. A control system containing glucose as carbon source was used to compare with treated and non-treated sludge samples. The reactor was maintained at 28° C. agitated only by recirculation. Samples were collected and analyzed every 12 hours for 3 days for COD and ethanol production.

The results show that ethanol production followed the growth curve of Saccharomyces sp. until stationary phase is reached. The ethanol therefore reached its maximum production after 48 hours after which production decreased.

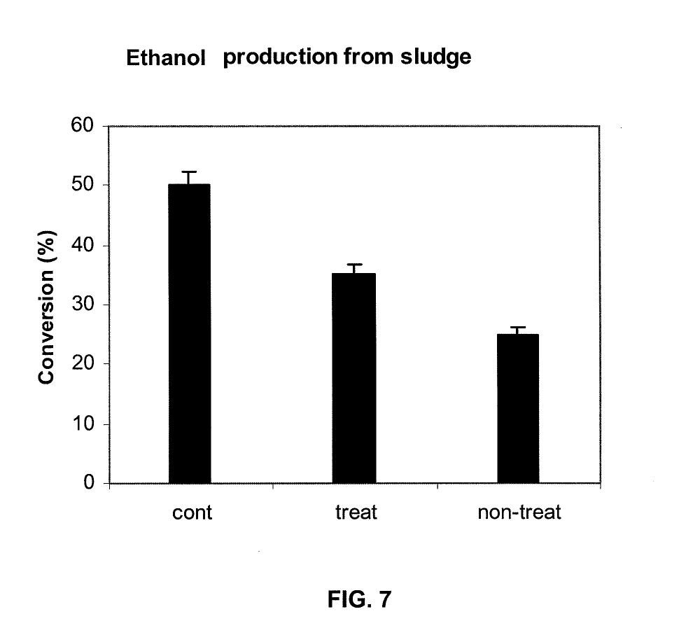

As can be observed in FIG. 7, after 48 hours of culture, approximately 30% and 20% of COD was converted to ethanol using the treated and untreated sludge as a carbon source (respectively). As a control, approximately 50% of COD was converted to ethanol using glucose as a carbon source.

These results indicate that the treatment of sludge to remove heavy metals increases the production of ethanol from the sludge, and that ethanol can be produced even from non-treated sludge, although at significantly lower levels.

### Example 4

### Testing of a Photo Bioreactor for the Production of Hydrogen and Biodiesel from the CO₂ Effluents

This example demonstrates that biodiesel and hydrogen are produced by microalgae cultures extracting the CO₂ from the effluent of the anaerobic bioreactor.

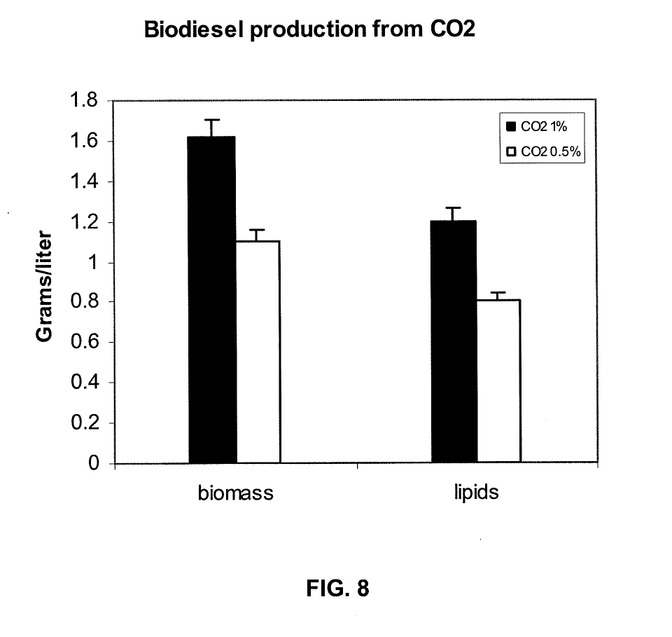

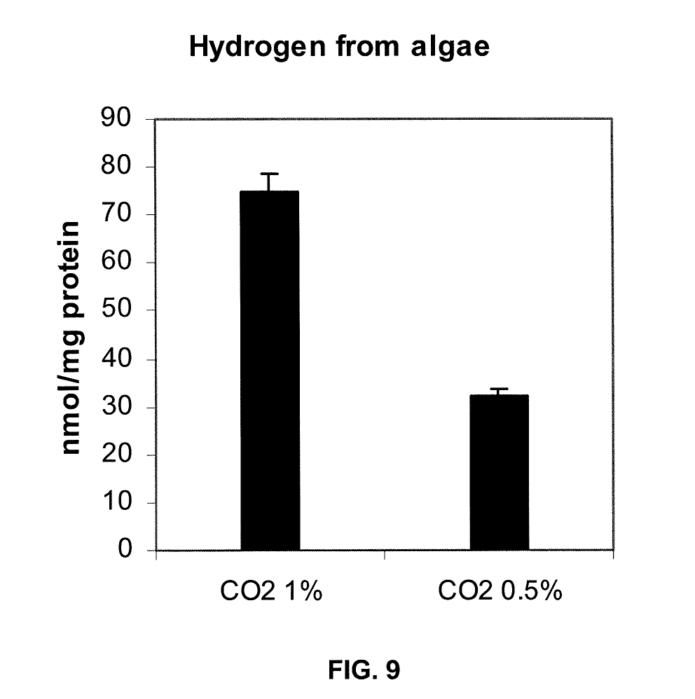

A culture of microalgae (Chlorella sp.) was used to assess cell-lipid accumulation and hydrogen production after capture of different CO₂ concentrations produced during sludge digestion of an anaerobic bioreactor. Chlorella sp. cells were grown in a 1-L bioreactor culture under constant illumination with a fluorescent lamp (15 Wm-2) at 28° C. The photobioreactor was bubbled with effluent gas from anaerobic bioreactor to reach 0.5 and 1.0% (v/v) levels of CO₂. The growth of culture was recorded at 5-day intervals. Chlorella sp. cells were harvested at the late logarithmic growth phase, collected and evaluated for biomass accumulation, lipid content and hydrogen production. FIG. 8 shows the performance of a biosynthetic multiphasic photosynthetic reactor in the production of biodiesel from CO₂ outlet from an anaerobic bioreactor. Performance was assessed by the yields of biomass according to the concentration of CO₂ injected. Substantial biomass is achieved at 1% and 0.5% CO₂ concentrations, with more biomass produced at 1%. The lipids extracted to produce biodiesel reached approximately 65% of the biomass produced at different concentration of CO₂. FIG. 9 shows the performance with respect to the production of hydrogen in relation to biomass (nmol hydrogen per gram of protein). The hydrogen release was approximately 76 nmol of hydrogen per gram of protein when biomass was grown at 1% of CO₂, while 32 nmol of hydrogen per gram of protein was produced when 0.5% CO₂ was injected.

These results demonstrate that the CO₂ effluent from a sludge-digesting anaerobic bioreactor can be captured and fixed to support the growing of microalgae cells in a photo bioreactor system. This photo bioreactor can be used efficiently to capture the CO₂ produced in the anaerobic reactors, before that CO₂ is disposed in the environment.

### REFERENCES

Ascon M A, Lebeault J M. (1999). High Efficiency of a coupled aerobic-anaerobic recycling biofilm reactor system in the degradation of recalcitrant chloroaromatic xenobiotic compounds. Appl. Microbiol. Biotechnol. 52(4):592-9.
Ascon M A, Thomas D, Lebeault J M. (1995). Activity of synchronized cells of a steady state biofilm recirculated reactor during xenobiotic biodegradation. Appl. Environ. Microbiol. 61(3):920-5.
Ascon M A, Ascon D B, Lebeault J M. (1995). Degradation activity of adhered and suspended Pseudomonas cells cultured on 2,4,6-trichlorophenol, measured by indirect conductimetry. J Appl Bacteriol. 79:617-624.
Tyagi, R. D., J. F. Blais, N. Meunier, and H. Benmoussa. (1997). Simultaneous sewage sludge digestion and metal leaching—Effect of sludge solids concentration. Water Res. 31:105-118.
Ryu, H. W., Y. J. Kim, K. S. Cho, K. S. Kang, and H. Choi. (1998). Effect of sludge concentration on removal of heavy metals from digested sludge by Thiobacillus ferrooxidans. Korean J. Biotechnol. Bioeng. 13:279-283.
Lettinga G, Hulshoff Pol L W. (1991). UASB process design for various types of wastewater. Water Sci Technol. 24: 87-107.
Seghezzo, L., Zeeman, G., van Lier, J. B., Hamelers, H. V. M. and Letting a, G. (1998). A review: the anaerobic treatment of sewage in UASB and EGSB reactors. Bioresource Technol., 65, 175-190.
Akkerman I, Janssen M, Rocha J, Wijffels R H. (2002). Photobiological hydrogen production: photochemical efficiency and bioreactor design. International Journal of Hydrogen Energy 27:1195-1208.
Xu, H.; Miao, X. and Q. Wu. (2006). High Quality Biodiesel Production from a Microalga Chlorella protothecoides by Heterotrophic Growth in Fermenters. Journal of Biotechnology. 126: 499-507.
Ballesteros, I., Oliva, J. M., Saez, F., Ballesteros, M. (2001). Ethanol production from lignocellulosic byproducts of olive oil extraction. Appl. Biochem. Biotechnol. 91-93, 237-252.
Fan Z, South C, Lyford K, Munsie J, van Walsum P, Lynd L R. (2003). Conversion of paper sludge to ethanol in a semicontinuous solids-fed reactor. 26:93-101.

1. A method for producing a biofuel or bioenergy product from municipal, industrial, and/or farm sewage sludge, comprising:
leaching metals from the sludge through the action of acid-producing, sulfur-oxidizing microorganisms, to thereby produce a treated sludge; and
synthesizing one or more biofuel or bioenergy products from the treated sludge by microbial action.

2. The method of claim 1, wherein the sludge is a recalcitrant sludge left from anaerobic and/or aerobic digestion of raw sludge.

3. The method of claim 1, wherein the recalcitrant sludge has undergone one or more of composting, drying, dewatering, thickening, pressing, filtering, centrifugation, ultraviolet or chemical disinfection, lime stabilization, and/or thermal processing.

4. The method of claim 1, wherein the sludge has a high content of heavy metals.

5. The method of claim 4, wherein the heavy metals are one or more of Zn, Pb, Cu, Cr, Ni, Cd, and Hg.

6. The method of claim 5, wherein at least one heavy metal is present in the sludge at more than 100 ppm.

7. The method of claim 6, wherein at least one heavy metal is present in the sludge at from about 400 to about 1000 ppm.

8. The method of claim 5, wherein the sludge is recalcitrant sludge contaminated with high levels of lead (Pb) and/or cadmium (Cd).

9. The method of claim 1, wherein the sludge has a high content of at least one bacterial, viral, and/or parasitic pathogen.

10. The method of claim 9, wherein the pathogen(s) include one or more pathogens selected from enteropathogenic E. coli, Salmonella, Shigella, Yersinia, Vibrio Cholerae, Cryptosporidium, Giardia, Entamoeba, Norovirus, and Rotavirus.

11. The method of claim 1, wherein the sludge is an industrial sludge containing one or more of a polychlorinated biphenyl (PCB), polycyclic aromatic hydrocarbon (PAH), dioxin, pesticide, endocrine disrupter, antibiotic, tannin, lignin, resin, terpene, chlorophenolic compound, alkyl-sulfonate, alkylphenol, oil, grease, heavy metals, ammonia, and aliphatic or aromatic hydrocarbon.

12. The method of claim 1, wherein the sludge is farm sludge comprising animal manure.

13. The method of claim 12, wherein the farm sludge is farmyard manure or farm slurry.

14. The method of claim 12, wherein the farm sludge comprises waste from swine, horse, cattle, sheep, and/or poultry.

15. The method of claim 12, wherein the farm sludge comprises pig waste.

16. The method of claim 1, wherein the sludge is supplemented with an aqueous phase containing a mineral salt medium.

17. The method of claim 16, wherein the mineral salt medium comprises a sulfur substrate for supporting the action of sulfur-oxidizing bacteria.

18. The method of claim 1, wherein the method operates in batch, semi-continuously, or continuously.

19. The method of claim 1, wherein the leaching of metals takes place in a bioleaching system comprising at least one continuous stirred-tank reactor.

20. The method of claim 1, wherein the metals are recovered after solubilization.

21. The method of claim 19, wherein the bioleaching system further comprises a tubular reactor.

22. The method of claim 1, wherein the bioleaching system obtains a sludge pH of from 1 to 4.

23. The method of claim 22, further comprising, separating the liquid phase from biomass when the pH is from 1 to 4, precipitating heavy metals from the liquid phase by restoring the pH; removing precipitated heavy metals; and then adding the liquid phase back to the biomass.

24. The method of claim 1, wherein the biofuel or bioenergy product is methane, hydrogen, methanol, ethanol, butanol, and/or biodiesel.

25. The method of claim 1, wherein the synthesis of biofuels takes place in at least one anaerobic reactor.

26. The method of claim 25, wherein the anaerobic reactor is a multiphasic bioreactor for production of methane.

27. The method of claim 25, wherein the anaerobic bioreactor is a USAB reactor or an EGSB reactor.

28. The method of claim 25, wherein the anaerobic bioreactor is a multiphasic bioreactor for the production of ethanol, butanol, or methanol.

29. The method of claim 1, wherein biofuel synthesis takes place in at least one photosynthesis bioreactor.

30. The method of claim 29, wherein the photosynthesis bioreactor is a multiphasic bioreactor having photosynthetic microorganisms forming biofilms on support surfaces.

31. The method of claim 29, wherein the photosynthetic microorganisms are supported by effluent CO₂ from an anaerobic bioreactor.

32. The method of claim 1, wherein the acid-producing, sulfur-oxidizing bacteria for bioleaching is/are listed in Table 1.

33. The method of claim 1, wherein the microorganism producing the biofuel or bioenergy product is/are listed in Table 2.

34. The method of claim 33, wherein the biofuel or bioenergy product is methane, and the methanogenic microorganisms are one or a consortium of Methanosarcina, Methanosaeta and/or Methanothrix species.

35. The method of claim 33, wherein the biofuel or bioenergy product is an alcohol, and the microorganisms is one or more fermentative microorganisms.

36. The method of claim 35, wherein the fermentative microorganism includes one or more Zymomonas sp. and/or Saccharomyces sp.

37. The method of claim 35, wherein the biofuel or bioenergy product is butanol, and the fermentative microorganisms include a Clostridium sp.

38. The method of claim 31, wherein the photosynthesis bioreactor includes one or a consortium of Synechococcus sp., Chlorella sp., Synechocystis sp., Nitzchia sp., and/or Schizochytriu sp.

39. The method of claim 31, wherein the photosynthesis bioreactor includes a cyanobacteria or algae.

40. The method of claim 1, wherein the method is performed at an industrial scale.

41. The method of claim 1, further comprising, recovering the biofuel or bioenergy product.

42. The method of claim 41, wherein the biofuel or bioenergy product is ethanol, methanol, and/or butanol, and the product is recovered from liquid material by a molecular sieve or distillation.

43. The method of claim 41, wherein biogas containing hydrogen and/or methane is drawn off from the system as pipeline gas.

44. The method of claim 41, wherein hydrogen and methane are purified from biogas.

45. An integrated system for converting municipal, industrial, or farm sewage sludge to one or more biofuel or bioenergy products, comprising:
a bioleaching system suitable for extracting heavy metals from sludge, to prepare a treated sludge; and
an anaerobic bioreactor operably connected to receive said treated sludge; and
optionally a photo bioreactor operably connected to receive gas effluent from the anaerobic bioreactor.

46. The integrated system of claim 45, wherein the bioleaching reactor contains an inlet for influent sewage sludge.

47. The integrated system of claim 45, wherein the bioleaching system contains a first outlet for effluent heavy metals, and a second outlet for effluent liquid having a reduced level of heavy metals.

48. The integrated system of claim 45, wherein the bioleaching system comprises a continuous stirred-tank reactor.

49. The integrated system of claim 48, wherein the continuous stirred-tank reactor is followed by a tubular reactor.

50. The integrated system of claim 45, wherein the bioleaching system comprises a recipient unit for receiving sludge having solubilized heavy metals, the recipient unit comprising a centrifuge.

51. The integrated system of claim 45, wherein the anaerobic bioreactor is a UASB reactor or a EGSB reactor.

52. The integrated system of claim 45, wherein the anaerobic bioreactor is a multiphasic bioreactor containing fermentative or methanogenic microbes forming biofilms on solid supports.

53. The integrated system of claim 45, wherein the anaerobic bioreactor contains an inlet for treated sludge from the bioleaching system.

54. The integrated system of claim 45, wherein the anaerobic bioreactor contains an in-series connection to transport gas effluent from the anaerobic reactor to the photo bioreactor.

55. The integrated system of claim 54, wherein the photo bioreactor contains an inlet for liquid media.

56. The integrated system of claim 54, wherein the anaerobic bioreactor further comprises an outlet for transporting the biofuels and bioenergy products.

57. The integrated system of claim 54, further comprising a feedback connection between the anaerobic bioreactor to the bioleaching reactor.

58. The integrated system of claim 45, further comprising a system of pumps and/or valves operably connecting inlets and outlets.

59. The integrated system of claim 45, wherein the working volume of the system is from about 100 gallons to about 100,000 gallons.
